(19) United States
(12) Patent Application Publication
(10) Pub. No.: US 2016/0153045 A1
(43) Pub. Date: Jun. 2, 2016

(54) Biomarkers for Increased Risk of Drug-Induced 5-Aminosalicylate Nephrotoxicity

(71) Applicant: Severe Adverse Event (SAE) Consortium, Chicago, IL (US)

(72) Inventors: Graham Alastair Richard HEAP, Exeter Devon (GB); Tariq AHMAD, Exeter Devon (GB)

(21) Appl. No.: 14/956,765

(22) Filed: Dec. 2, 2015

Related U.S. Application Data
(60) Provisional application No. 62/086,576, filed on Dec. 2, 2014.

(51) Int. Cl. C12Q 1/68

(52) U.S. Cl. CPC C12Q 1/6883; C12Q 2600/156; C12Q 2600/172; C12Q 2600/106

(57) ABSTRACT

The present invention provides a method for predicting the risk of a patient for developing adverse drug reactions, particularly 5-aminosalicylate nephrotoxicity (5-ASA nephrotoxicity). The invention also provides a method of identifying a subject afflicted with, or at risk of, developing 5-ASA nephrotoxicity. In some aspects, the methods comprise analyzing at least one genetic marker, wherein the presence of the at least one genetic marker indicates that the subject is afflicted with, or at risk of, developing 5-ASA nephrotoxicity.

This application claims the benefit of priority of U.S. Provisional Application Ser. No. 62/086,576, filed Dec. 2, 2014, the contents of which are hereby incorporated by reference in its entirety.

### TECHNICAL FIELD

The present disclosure relates generally to methods for identifying genetic risk factors for adverse reactions to drugs. More specifically, the present disclosure relates to methods for predicting what drugs will cause 5-aminosalicylate nephrotoxicity, and in which patients.

### BACKGROUND

Adverse reactions to drugs are a major cause of morbidity and death. Frequently occurring adverse drug reactions include 5-aminosalicylate nephrotoxicity (5-ASA nephrotoxicity). One of the most commonly used class of agents to maintain and induce remission in patients with inflammatory bowel disease (Crohn's disease or ulcerative colitis) are the 5-aminosalicylate drugs (sulfasalazine, mesalazine, balsalazide, olsalazine). Exposure to these agents has been associated with the development of an idiosyncratic nephrotoxicity.

Common drugs that have been associated with 5-ASA nephrotoxicity include sulfasalazine, mesalazine, balsalazide, and olsalazine.

There is a need for markers that can predict the existence of or predisposition to 5-ASA nephrotoxicity. Several studies have identified genetic risk factors for drug-related severe adverse events. However, there is currently no clinically useful method for predicting what drugs will cause 5-ASA nephrotoxicity and in which patients.

### SUMMARY

An aspect of the invention provides a method for predicting the risk of a patient for developing adverse drug reactions, particularly 5-aminosalicylate nephrotoxicity (5-ASA nephrotoxicity).

5-ASA nephrotoxicity may be caused by the 5-aminosalicylate class of drugs such as sulfasalazine, mesalazine, balsalazide, and olsalazine.

Another aspect of the invention provides a method of identifying a subject afflicted with, or at risk of, developing 5-ASA nephrotoxicity comprising (a) obtaining a nucleic acid-containing sample from the subject; and (b) analyzing the sample to detect the presence of at least one genetic marker, wherein the presence of the at least one genetic marker indicates that the subject is afflicted with, or at risk of, developing 5-ASA nephrotoxicity. The method may further comprise treating the subject based on the results of step (b). The method may further comprise taking a clinical history from the subject. Genetic markers that are useful for the invention include, but are not limited to, alleles, microsatellites, SNPs, and haplotypes. The sample may be any sample capable of being obtained from a subject, including but not limited to blood, sputum, saliva, mucosal scraping and tissue biopsy samples.

In some embodiments of the invention, the genetic markers are SNPs selected from those listed in Table 1. In other embodiments, genetic markers that are linked to each of the SNPs can be used to predict the corresponding 5-ASA nephrotoxicity risk.

The presence of the genetic marker can be detected using any method known in the art. Analysis may comprise nucleic acid amplification, such as PCR. Analysis may also comprise primer extension, restriction digestion, sequencing, hybridization, a DNAse protection assay, mass spectrometry, labeling, and separation analysis.

Other features and advantages of the disclosure will be apparent from the detailed description, drawings and from the claims.

### BRIEF DESCRIPTION OF THE DRAWINGS

FIG. 1 shows a Manhattan Plot depicting the association of each tested SNP with nephrotoxicity to 5-ASA agents for all samples tested.

FIG. 2 shows a Manhattan Plot depicting association of each tested SNP with nephrotoxicity to 5-ASA agents when only those samples with a diagnosis of interstitial nephritis on renal biopsy are included to refine the phenotype definition.

FIG. 3 shows boxplots for the cohort of 151 5-ASA induced nephrotoxicity cases demonstrating time from treatment commencement with 5-ASA to development of nephrotoxicity and subsequent recovery. Serial boxplots demonstrating the median creatinine levels for n=151 patients taken at each of the 5 time study points detailed in the Methods (note n=92 for Last normal group). The position of the boxplot on the x-axis corresponds to the median time point from starting 5-ASA at which these creatinine values were obtained (the width of the boxplot is the 95% confidence interval of this value). The drug was started at Time point 0 for all patients. The arrow labelled “Stop” illustrates the median time at which the agent was stopped after commencement (with the 95% CI of this value illustrated with the grey box). Outlier values have not been visualised. Patients who underwent renal replacement therapy were excluded from the “Best Recovered” group.

FIG. 4 shows time from initiation of 5-ASA agents to the development of nephrotoxicity and recovery of renal function to baseline level. FIG. 4 displays the time in days from initiation of 5-ASA agents to the development of nephrotoxicity by the recovery status. The horizontal line illustrates the median for each cohort.

FIGS. 5A-B show genome wide Manhattan plot (including HLA imputation) Blue line b (P=1×10⁻⁵), Red line a (P=5×10⁻⁸) (FIG. 5A) and QQ plot (including HLA-imputation) (FIG. 5B).

FIGS. 6A-B show genome wide Manhattan plot (including HLA imputation) excluding cases that did not have a renal biopsy demonstrating interstitial nephritis (n=55) for all SNPs with a MAF>0.05. Blue line b (P=1×10⁻⁵), Red line a (P=5×10⁻⁸) (FIG. 6A) and QQ plot (including HLA imputation) excluding cases that did not have a renal biopsy demonstrating interstitial nephritis (n=55) (FIG. 6B).

### DETAILED DESCRIPTION

For the purposes of promoting an understanding of the principles of the invention, reference will now be made to specific embodiments and specific language will be used to describe the same. It will nevertheless be understood that no limitation of the scope of the invention is thereby intended, and that such alterations and further modifications of the invention, and such further applications of the principles of the invention as illustrated herein as would normally occur to one skilled in the art to which the invention relates, are contemplated as within the scope of the invention.

All terms as used herein are defined according to the ordinary meanings they have acquired in the art. Such definitions can be found in any technical dictionary or reference known to the skilled artisan, such as the McGraw-Hill Dictionary of Scientific and Technical Terms (McGraw-Hill, Inc.), Molecular Cloning: A Laboratory Manual (Cold Springs Harbor, N.Y.), Remington's Pharmaceutical Sciences (Mack Publishing, PA), and Stedman's Medical Dictionary (Williams and Wilkins, MD). These references, along with those references, patents, and patent applications cited herein are hereby incorporated by reference in their entirety.

The term “marker” as used herein refers to any morphological, biochemical, or nucleic acid-based phenotypic difference which reveals a DNA polymorphism. The presence of markers in a sample may be useful to determine the phenotypic status of a subject (e.g., whether an individual has or has not been afflicted with 5-ASA nephrotoxicity), or may be predictive of a physiological outcome (e.g., whether an individual is likely to develop 5-ASA nephrotoxicity). The markers may be differentially present in a biological sample or fluid, such as blood plasma or serum. The markers may be isolated by any method known in the art, including methods based on mass, binding characteristics, or other physicochemical characteristics. As used herein, the term “detecting” includes determining the presence, the absence, or a combination thereof, of one or more markers.

Non-limiting examples of nucleic acid-based, genetic markers include alleles, microsatellites, single nucleotide polymorphisms (SNPs), haplotypes, copy number variants (CNVs), insertions, and deletions.

The term “allele” as used herein refers to an observed class of DNA polymorphism at a genetic marker locus. Alleles may be classified based on different types of polymorphism, for example, DNA fragment size or DNA sequence. Individuals with the same observed fragment size or same sequence at a marker locus have the same genetic marker allele and thus are of the same allelic class.

The term “locus” as used herein refers to a genetically defined location for a collection of one or more DNA polymorphisms revealed by a morphological, biochemical or nucleic acid-bred analysis.

The term “genotype” as used herein refers to the allelic composition of an individual at genetic marker loci under study, and “genotyping” refers to the process of determining the genetic composition of individuals using genetic markers.

The term “single nucleotide polymorphism” (SNP) as used herein refers to a DNA sequence variation occurring when a single nucleotide in the genome or other shared sequence differs between members of a species or between paired chromosomes in an individual. The difference in the single nucleotide is referred to as an allele. A “haplotype” as used herein refers to a set of single SNPs on a single chromatid that are statistically associated.

The term “microsatellite” as used herein refers to polymorphic loci present in DNA that comprise repeating units of 1-6 base pairs in length.

An aspect of the invention provides a method for predicting the risk of a patient for developing adverse drug reactions, particularly 5-ASA nephrotoxicity. As used herein, an “adverse drug reaction” is as an undesired and unintended effect of a drug. A “drug” as used herein is any compound or agent that is administered to a patient for prophylactic, diagnostic or therapeutic purposes.

5-ASA nephrotoxicity may be caused by the 5-aminosalicylate class of drugs such as sulfasalazine, mesalazine, balsalazide, and olsalazine.

Another aspect of the invention provides a method of identifying a subject afflicted with or at risk of developing 5-ASA nephrotoxicity comprising (a) obtaining a nucleic acid-containing sample from the subject; and (b) analyzing the sample to detect the presence of at least one genetic marker, wherein the presence of the at least one genetic marker indicates that the subject is afflicted with or at risk of developing 5-ASA nephrotoxicity. The method may further comprise treating the subject based on the results of step (b). The method may further comprise taking a clinical history from the subject. Genetic markers that are useful for the invention include, but are not limited to, alleles, microsatellites, SNPs, haplotypes, CNVs, insertions, and deletions.

In some embodiments of the invention, the genetic markers are one or more SNPs selected from those listed in Table 1.

Each person's genetic material contains a unique SNP pattern that is made up of many different genetic variations. SNPs may serve as biological markers for pinpointing a disease on the human genome map, because they are usually located near a gene found to be associated with a certain disease. Occasionally, a SNP may actually cause a disease and, therefore, can be used to search for and isolate the disease-causing gene.

In accordance with the invention, at least one marker may be detected. It is to be understood, and is described herein, that one or more markers may be detected and subsequently analyzed, including several or all of the markers identified. Further, it is to be understood that the failure to detect one or more of the markers of the invention, or the detection thereof at levels or quantities that may correlate with 5-ASA nephrotoxicity, may be useful as a means of selecting the individuals afflicted with or at risk for developing 5-ASA nephrotoxicity, and that the same forms a contemplated aspect of the invention.

In addition to the SNPs listed in Table 1, genetic markers that are linked to each of the SNPs may be used to predict the corresponding 5-ASA nephrotoxicity risk as well. The presence of equivalent genetic markers may be indicative of the presence of the allele or SNP of interest, which, in turn, is indicative of a risk for 5-ASA nephrotoxicity. For example, equivalent markers may co-segregate or show linkage disequilibrium with the marker of interest. Equivalent markers may also be alleles or haplotypes based on combinations of SNPs.

The equivalent genetic marker may be any marker, including alleles, microsatellites, SNPs, and haplotypes. In some embodiments, the useful genetic markers are about 200 kb or less from the locus of interest. In other embodiments, the markers are about 100 kb, 80 kb, 60 kb, 40 kb, or 20 kb or less from the locus of interest.

To further increase the accuracy of risk prediction, the marker of interest and/or its equivalent marker may be determined along with the markers of accessory molecules and co-stimulatory molecules which are involved in the interaction between antigen-presenting cell and T-cell interaction. For example, the accessory and co-stimulatory molecules include cell surface molecules (e.g., CD80, CD86, CD28, CD4, CD8, T cell receptor (TCR), ICAM-1, CD11a, CD58, CD2, etc.), and inflammatory or pro-inflammatory cytokines, chemokines (e.g., TNF-α), and mediators (e.g., complements, apoptosis proteins, enzymes, extracellular matrix components, etc.). Also of interest are genetic markers of drug metabolizing enzymes which are involved in the bioactivation and detoxification of drugs. Non-limiting examples of drug metabolizing enzymes include phase I enzymes (e.g., cytochrome P450 superfamily), and phase II enzymes (e.g., microsomal epoxide hydrolase, arylamine N-acetyltransferase, UDP-glucuronosyl-transferase, etc.).

Another aspect of the invention provides a method for pharmacogenomic profiling. Accordingly, a panel of genetic factors is determined for a given individual, and each genetic factor is associated with the predisposition for a disease or medical condition, including adverse drug reactions. In some embodiments, the panel of genetic factors may include at least one SNP selected from Table 1. The panel may include equivalent markers to the markers in Table 1. The genetic markers for accessory molecules, co-stimulatory molecules and/or drug metabolizing enzymes described above may also be included.

Yet another aspect of the invention provides a method of screening and/or identifying agents that can be used to treat 5-ASA nephrotoxicity by using any of the genetic markers of the invention as a target in drug development. For example, cells expressing any of the SNPs or equivalents thereof may be contacted with putative drug agents, and the agents that bind to the SNP or equivalent are likely to inhibit the expression and/or function of the SNP. The efficacy of the candidate drug agent in treating 5-ASA nephrotoxicity may then be further tested.

In some embodiments, it may be useful to amplify the target sequence before evaluating the genetic marker. Nucleic acids used as a template for amplification may be isolated from cells, tissues or other samples according to standard methodologies such as are described, for example, in Sambrook et al., 1989. In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA. The DNA also may be from a cloned source or synthesized in vitro.

The term “primer,” refers to any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form.

For amplification of SNPs, pairs of primers designed to selectively hybridize to nucleic acids flanking the polymorphic site may be contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex may be contacted with one or more enzymes that facilitate template-dependent nucleic acid synthesis. Multiple rounds of amplification, also referred to as “cycles,” are conducted until a sufficient amount of amplification product is produced.

It is also possible that multiple target sequences will be amplified in a single reaction. Primers designed to expand specific sequences located in different regions of the target genome, thereby identifying different polymorphisms, would be mixed together in a single reaction mixture. The resulting amplification mixture would contain multiple amplified regions, and could be used as the source template for polymorphism detection using the methods described in this application.

Any known template dependent process may be advantageously employed to amplify the oligonucleotide sequences present in a given template sample. One of the best known amplification methods is the polymerase chain reaction (PCR), which is described in U.S. Pat. Nos. 4,683,195, 4,683,202 and 4,800,159, and in Innis et al., 1988, each of which is incorporated herein by reference in their entirety.

A reverse transcriptase PCR amplification procedure may be performed when the source of nucleic acid is fractionated or whole cell RNA. Methods of reverse transcribing RNA into cDNA are well known and are described in, for example, Sambrook et al., 1989. Alternative exemplary methods for reverse polymerization utilize thermostable DNA polymerases. These methods are described, for example, in International Publication WO 90/07641. Polymerase chain reaction methodologies are well known in the art. Representative methods of RT-PCR are described, for example, in U.S. Pat. No. 5,882,864.

Another method for amplification is ligase chain reaction (LCR), disclosed, for example, in European Application No. 320 308, incorporated herein by reference in its entirety. U.S. Pat. No. 4,883,750 describes a method similar to LCR for binding probe pairs to a target sequence. A method based on PCR and oligonucleotide ligase assay (OLA), disclosed, for example, in U.S. Pat. No. 5,912,148, may also be used.

Another ligase-mediated reaction is disclosed by Guilfoyle et al. (1997). Genomic DNA is digested with a restriction enzyme and universal linkers are then ligated onto the restriction fragments. Primers to the universal linker sequence are then used in PCR to amplify the restriction fragments. By varying the conditions of the PCR, one can specifically amplify fragments of a certain size (e.g., fewer than 1000 bases). A benefit to using this approach is that each individual region would not have to be amplified separately. There would be the potential to screen thousands of SNPs from the single PCR reaction.

Q-beta Replicase, described, for example, in International Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence, which may then be detected.

An isothermal amplification method, in which restriction endonucleases and ligases are used to achieve the amplification of target molecules that contain nucleotide 5′-[alpha-thio]riphosphates in one strand of a restriction site may also be useful in the amplification of nucleic acids in the present invention (Walker et al., 1992). Strand Displacement Amplification (SDA), disclosed, for example, in U.S. Pat. No. 5,916,779, is another method of carrying out isothermal amplification of nucleic acids which involves multiple rounds of strand displacement and synthesis, e.g., nick translation.

Other nucleic acid amplification procedures include polymerization-based amplification systems (TAS), for example, nucleic acid sequence based amplification (NASBA) and 3SR (Kwoh et al., 1989; International Application WO 88/10315, incorporated herein by reference in their entirety). European Application No. 329 822 discloses a nucleic acid amplification process involving cyclically synthesizing single-stranded RNA (ssRNA), ssDNA, and double-stranded DNA (dsDNA), which may be used in accordance with the present invention.

International Application WO 89/06700 discloses a nucleic acid sequence amplification scheme based on the hybridization of a promoter region/primer sequence to a target single-stranded DNA (ssDNA) followed by polymerization of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

### Methods of Detection

The genetic markers of the invention may be detected using any method known in the art. For example, genomic DNA may be hybridized to a probe that is specific for the allele of interest. The probe may be labeled for direct detection, or contacted by a second, detectable molecule that specifically binds to the probe. Alternatively, cDNA, RNA, or the protein product of the allele may be detected. For example, serotyping or microcytotoxity methods may be used to determine the protein product of the allele. Similarly, equivalent genetic markers may be detected by any methods known in the art.

It is within the purview of one of skill in the art to design genetic tests to screen for 5-ASA nephrotoxicity or a predisposition for 5-ASA nephrotoxicity based on analysis of the genetic markers of the invention. For example, a genetic test may be based on the analysis of DNA for SNP patterns. Samples may be collected from a group of individuals affected by 5-ASA nephrotoxicity due to drug treatment and the DNA analyzed for SNP patterns. Non-limiting examples of sample sources include blood, sputum, saliva, mucosal scraping or tissue biopsy samples. These SNP patterns may then be compared to patterns obtained by analyzing the DNA from a group of individuals unaffected by 5-ASA nephrotoxicity due to drug treatment. This type of comparison, called an “association study,” can detect differences between the SNP patterns of the two groups, thereby indicating which pattern is most likely associated with 5-ASA nephrotoxicity. Eventually, SNP profiles that are characteristic of a variety of diseases will be established. These profiles can then be applied to the population at general, or those deemed to be at particular risk of developing 5-ASA nephrotoxicity.

Various techniques may be used to assess genetic markers. Non-limiting examples of a few of these techniques are discussed here and also described in US Patent Publication 2007/026827, the disclosure of which is herein incorporated by reference in its entirety. In accordance with the invention, any of these methods may be used to design genetic tests for affliction with or predisposition to 5-ASA nephrotoxicity. Additionally, these methods are continually being improved and new methods are being developed. It is contemplated that one of skill in the art will be able to use any improved or new methods, in addition to any existing method, for detecting and analyzing the genetic markers of the invention.

Restriction Fragment Length Polymorphism (RFLP) is a technique in which different DNA sequences may be differentiated by analysis of patterns derived from cleavage of that DNA. If two sequences differ in the distance between sites of cleavage of a particular restriction endonuclease, the length of the fragments produced will differ when the DNA is digested with a restriction enzyme. The similarity of the patterns generated can be used to differentiate species (and even individual species members) from one another.

Restriction endonucleases are the enzymes that cleave DNA molecules at specific nucleotide sequences depending on the particular enzyme used. Enzyme recognition sites are usually 4 to 6 base pairs in length. Generally, the shorter the recognition sequence, the greater the number of fragments generated. If molecules differ in nucleotide sequence, fragments of different sizes may be generated. The fragments can be separated by gel electrophoresis. Restriction enzymes are isolated from a wide variety of bacterial genera and are thought to be part of the cell's defenses against invading bacterial viruses. Use of RFLP and restriction endonucleases in genetic marker analysis, such as SNP analysis, requires that the SNP affect cleavage of at least one restriction enzyme site.

Primer Extension is a technique in which the primer and no more than three NTPs may be combined with a polymerase and the target sequence, which serves as a template for amplification. By using fewer than all four NTPs, it is possible to omit one or more of the polymorphic nucleotides needed for incorporation at the polymorphic site. The amplification may be designed such that the omitted nucleotide(s) is(are) not required between the 3′ end of the primer and the target polymorphism. The primer is then extended by a nucleic acid polymerase, such as Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present.

Oligonucleotide Hybridization is a technique in which oligonucleotides may be designed to hybridize directly to a target site of interest. The hybridization can be performed on any useful format. For example, oligonucleotides may be arrayed on a chip or plate in a microarray. Microarrays comprise a plurality of oligos spatially distributed over, and stably associated with, the surface of a substantially planar substrate, e.g., a biochip. Microarrays of oligonucleotides have been developed and find use in a variety of applications, such as screening and DNA sequencing.

In gene analysis with microarrays, an array of “probe” oligonucleotides is contacted with a nucleic acid sample of interest, i.e., a target. Contact is carried out under hybridization conditions and unbound nucleic acid is then removed. The resultant pattern of hybridized nucleic acid provides information regarding the genetic profile of the sample tested. Methodologies of gene analysis on microarrays are capable of providing both qualitative and quantitative information.

A variety of different arrays which may be used is known in the art. The probe molecules of the arrays which are capable of sequence-specific hybridization with target nucleic acid may be polynucleotides or hybridizing analogues or mimetics thereof, including: nucleic acids in which the phosphodiester linkage has been replaced with a substitute linkage, such as phosphorothioate, methylimino, methylphosphonate, phosphoramidate, guanidine and the like; and nucleic acids in which the ribose subunit has been substituted, e.g., hexose phosphodiester, peptide nucleic acids, and the like. The length of the probes will generally range from 10 to 1000 nts, wherein in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

Probe molecules arrayed on the surface of a substrate may correspond to selected genes being analyzed and be positioned on the array at a known location so that positive hybridization events may be correlated to expression of a particular gene in the physiological source from which the target nucleic acid sample is derived. The substrate with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. Different array configurations and methods for their production and use are known to those of skill in the art and disclosed, for example, in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755, the disclosures of which are herein incorporated by reference in their entireties.

Following hybridization, where non-hybridized labeled nucleic acid is capable of emitting a signal during the detection step, a washing step is employed in which unhybridized labeled nucleic acid is removed from the support surface, generating a pattern of hybridized nucleic acid on the substrate surface. Various wash solutions and protocols for their use are known to those of skill in the art and may be used.

Where the label on the target nucleic acid is not directly detectable, the array comprising bound target may be contacted with the other member(s) of the signal producing system that is being employed. For example, where the target is biotinylated, the array may be contacted with streptavidin-fluorescer conjugate under conditions sufficient for binding between the specific binding member pairs to occur. Following contact, any unbound members of the signal producing system will then be removed, e.g., by washing. The specific wash conditions employed will depend on the specific nature of the signal producing system that is employed, as will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, the potential for a mismatch hybridization event that could potentially generate a false positive signal on the pattern may be reduced by treating the array of hybridized target/probe complexes with an endonuclease under conditions sufficient such that the endonuclease degrades single stranded, but not double stranded, DNA. Various different endonucleases are known and may be used, including but not limited to mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described herein, the resultant hybridization pattern may be detected. In detecting or visualizing the hybridization pattern, the intensity or signal value of the label may also be quantified, such that the signal from each spot of the hybridization will be measured and compared to a unit value corresponding the signal emitted by known number of labeled target nucleic acids to obtain a count or absolute value of the copy number of each end-labeled target that is hybridized to a particular spot on the array in the hybridization pattern.

It will be appreciated that any useful system for detecting nucleic acids may be used in accordance with the invention. For example, mass spectrometry, hybridization, sequencing, labeling, and separation analysis may be used individually or in combination, and may also be used in combination with other known methods of detecting nucleic acids.

Electrospray ionization (ESI) is a type of mass spectrometry that is used to produce gaseous ions from highly polar, mostly nonvolatile biomolecules, including lipids. The sample is typically injected as a liquid at low flow rates (1-10 μL/min) through a capillary tube to which a strong electric field is applied. The field charges the liquid in the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice. A potential difference of between 1 to 5 kV (but more typically 2 to 3 kV) is applied to the capillary by power supply to generate a high electrostatic field (10⁶ to 10⁷ V/m) at the capillary tip. A sample liquid, carrying the analyte to be analyzed by the mass spectrometer, is delivered to the tip through an internal passage from a suitable source (such as from a chromatograph or directly from a sample solution via a liquid flow controller). By applying pressure to the sample in the capillary, the liquid leaves the capillary tip as small highly electrically charged droplets and further undergoes desolvation and breakdown to form single or multi-charged gas phase ions in the form of an ion beam. The ions are then collected by the grounded (or oppositely-charged) interface plate and led through an the orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258.

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals and bioactive peptides.

Secondary ion mass spectroscopy (SIMS) is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analyzed by the mass spectrometer in this method.

Laser desorption mass spectroscopy (LD-MS) involves the use of a pulsed laser, which induces desorption of sample material from a sample site, and effectively, vaporizes sample off of the sample substrate. This method is usually used in conjunction with a mass spectrometer, and can be performed simultaneously with ionization by adjusting the laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments is due to their different sizes causing different velocities. Each ion mass will thus have a different flight-time to the detector.

Other advantages of the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive and robust, and has been shown to be capable of detecting trace levels in natural samples without any prior extraction preparations.

Matrix Assisted Laser Desorption/Ionization Time-of Flight (MALDI-TOF) is a type of mass spectrometry useful for analyzing molecules across an extensive mass range with high sensitivity, minimal sample preparation and rapid analysis times. MALDI-TOF also enables non-volatile and thermally labile molecules to be analyzed with relative ease. One important application of MALDI-TOF is in the area of quantification of peptides and proteins, such as in biological tissues and fluids.

Surface Enhanced Laser Desorption and Ionization (SELDI) is another type of desorption/ionization gas phase ion spectrometry in which an analyte is captured on the surface of a SELDI mass spectrometry probe. There are several known versions of SELDI.

One version of SELDI is affinity capture mass spectrometry, also called Surface-Enhanced Affinity Capture (SEAC). This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” The capture reagent may be any material capable of binding an analyte. The capture reagent may be attached directly to the substrate of the selective surface, or the substrate may have a reactive surface that carries a reactive moiety that is capable of binding the capture reagent, e.g., through a reaction forming a covalent or coordinate covalent bond. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

Another version of SELDI is Surface-Enhanced Neat Desorption (SEND), which involves the use of probes comprising energy absorbing molecules that are chemically bound to the probe surface. Energy absorbing molecules (EAM) refer to molecules that are capable of absorbing energy from a laser desorption/ionization source and, thereafter, of contributing to desorption and ionization of analyte molecules in contact therewith. The EAM category includes molecules used in MALDI, frequently referred to as “matrix,” and is exemplified by cinnamic acid derivatives such as sinapinic acid (SPA), cyano-hydroxy-cinnamic acid (CHCA) and dihydroxybenzoic acid, ferulic acid, and hydroxyaceto-phenone derivatives. In certain versions, the energy absorbing molecule is incorporated into a linear or cross-linked polymer, e.g., a polymethacrylate. For example, the composition may be a co-polymer of α-cyano-4-methacryloyloxycinnamic acid and acrylate. In another version, the composition may be a co-polymer of α-cyano-4-methacryloyloxycinnamic acid, acrylate and 3-(tri-ethoxy)silyl propyl methacrylate. In another version, the composition may be a co-polymer of α-cyano-4-methacryloyloxycinnamic acid and octadecylmethacrylate (“C18 SEND”).

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light. SEPAR and other forms of SELDI are readily adapted to detecting a marker or marker profile, in accordance with the present invention.

In accordance with the invention, nucleic acid hybridization is another useful method of analyzing genetic markers. Nucleic acid hybridization is generally understood as the ability of a nucleic acid to selectively form duplex molecules with complementary stretches of DNAs and/or RNAs. Depending on the application, varying conditions of hybridization may be used to achieve varying degrees of selectivity of the probe or primers for the target sequence.

Typically, a probe or primer of between 10 and 100 nucleotides, and up to 1-2 kilobases or more in length, will allow the formation of a duplex molecule that is both stable and selective. Molecules having complementary sequences over contiguous stretches greater than 20 bases in length may be used to increase stability and selectivity of the hybrid molecules obtained. Nucleic acid molecules for hybridization may be readily prepared, for example, by directly synthesizing the fragment by chemical means or by introducing selected sequences into recombinant vectors for recombinant production.

For applications requiring high selectivity, relatively high stringency conditions may be used to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated by those of skill depending on the desired results.

It is within the purview of the skilled artisan to design and select the appropriate primers, probes, and enzymes for any of the methods of genetic marker analysis. For example, for detection of SNPs, the skilled artisan will generally use agents that are capable of detecting single nucleotide changes in DNA. These agents may hybridize to target sequences that contain the change. Or, these agents may hybridize to target sequences that are adjacent to (e.g., upstream or 5′ to) the region of change.

In general, it is envisioned that the probes or primers described herein will be useful as reagents in solution hybridization for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest, as described herein, is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section are incorporated herein by reference.

The synthesis of oligonucleotides for use as primers and probes is well known to those of skill in the art. Chemical synthesis can be achieved, for example, by the diester method, the triester method, the polynucleotide phosphorylase method and by solid-phase chemistry. Various mechanisms of oligonucleotide synthesis have been disclosed, for example, in U.S. Pat. Nos. 4,659,774, 4,816,571, 5,141,813, 5,264,566, 4,959,463, 5,428,148, 5,554,744, 5,574,146, and 5,602,244, each of which is incorporated herein by reference in its entirety.

In certain embodiments, nucleic acid products are separated by agarose, agarose-acrylamide or polyacrylamide gel electrophoresis using standard methods such as those described, for example, in Sambrook et al., 1989. Separated products may be cut out and eluted from the gel for further manipulation. Using low melting point agarose gels, the skilled artisan may remove the separated band by heating the gel, followed by extraction of the nucleic acid.

Separation of nucleic acids may also be effected by chromatographic techniques known in the art. There are many kinds of chromatography that may be used in the practice of the present invention, non-limiting examples of which include capillary adsorption, partition, ion-exchange, hydroxylapatite, molecular sieve, reverse-phase, column, paper, thin-layer, and gas chromatography, as well as HPLC.

A number of the above separation platforms may be coupled to achieve separations based on two different properties. For example, some of the primers may be coupled with a moiety that allows affinity capture, and some primers remain unmodified. Modifications may include a sugar (for binding to a lectin column), a hydrophobic group (for binding to a reverse-phase column), biotin (for binding to a streptavidin column), or an antigen (for binding to an antibody column). Samples may be run through an affinity chromatography column. The flow-through fraction is collected, and the bound fraction eluted (by chemical cleavage, salt elution, etc.). Each sample may then be further fractionated based on a property, such as mass, to identify individual components.

In certain aspects, it will be advantageous to employ nucleic acids of defined sequences of the present invention in combination with an appropriate means, such as a label, for determining hybridization. Various appropriate indicator means are known in the art, including fluorescent, radioactive, enzymatic or other ligands, such as avidin/biotin, which are capable of being detected. In the case of enzyme tags, colorimetric indicator substrates are known that may be employed to provide a detection means that is visibly or spectrophotometrically detectable, to identify specific hybridization with complementary nucleic acid containing samples. In yet other embodiments, the primer has a mass label that can be used to detect the molecule amplified. Other embodiments also contemplate the use of Taqman™ and Molecular Beacon™ probes.

Radioactive isotopes useful for the invention include, but are not limited to, tritium, ¹⁴C and ³²P. Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

The choice of label may vary, depending on the method used for analysis. When using capillary electrophoresis, microfluidic electrophoresis, HPLC, or LC separations, either incorporated or intercalated fluorescent dyes may be used to label and detect the amplification products. Samples are detected dynamically, in that fluorescence is quantitated as a labeled species moves past the detector. If an electrophoretic method, HPLC, or LC is used for separation, products can be detected by absorption of UV light. If polyacrylamide gel or slab gel electrophoresis is used, the primer for the extension reaction can be labeled with a fluorophore, a chromophore or a radioisotope, or by associated enzymatic reaction. Alternatively, if polyacrylamide gel or slab gel electrophoresis is used, one or more of the NTPs in the extension reaction can be labeled with a fluorophore, a chromophore or a radioisotope, or by associated enzymatic reaction. Enzymatic detection involves binding an enzyme to a nucleic acid, e.g., via a biotin:avidin interaction, following separation of the amplification products on a gel, then detection by chemical reaction, such as chemiluminescence generated with luminol. A fluorescent signal may be monitored dynamically. Detection with a radioisotope or enzymatic reaction may require an initial separation by gel electrophoresis, followed by transfer of DNA molecules to a solid support (blot) prior to analysis. If blots are made, they can be analyzed more than once by probing, stripping the blot, and then reprobing. If the extension products are separated using a mass spectrometer, no label is required because nucleic acids are detected directly.

While whole genome association (WGA) studies allow examination of many common SNPs in different individuals to identify associations between SNPs and traits like major diseases, exome sequencing studies can increase efficiency by allowing selective sequencing of at least the coding regions (i.e., the exons that are translated into proteins) of the genome, in which most functional variation is thought to occur. Some benefits of exome sequencing can include the detection of traits without traditional genetic linkage, with fewer available case studies (e.g., rare Mendelian diseases), with causal variants in different genes (i.e., genetic heterogeneity), and with diverse clinical features (i.e., phenotypic heterogeneity). The exome constitutes only about 1% of the entire human genome, and a large number of rare mutations have weak or no effects in non-coding sequences.

Target-enrichment methods like direct genomic selection (DGS) allow selective capture of genomic regions of interest from a DNA sample prior to sequencing. Other target-enrichment methods can include, but are not limited to, at least one of polymerase chain reaction (PCR) to amplify target-specific DNA sequences; molecular inversion probes of single-stranded DNA oligonucleotides that undergo an enzymatic reaction with target-specific DNA sequences to form circular DNA fragments; hybrid capture microarrays that contain fixed, tiled single-stranded DNA oligonucleotides with target-specific DNA sequences to hybridize sheared double-stranded fragments of genomic DNA; in-solution capture with single-stranded DNA oligonucleotides with target-specific DNA sequences synthesized in solution to hybridize sheared double-stranded fragments of genomic DNA in the solution; and methods using sequencing platforms, such as Sanger sequencing, 454™ sequencing (available from Roche Diagnostics Corp. (Branford, Conn.)), the Genome Analyzer™ (available from Illumina, Inc. (San Diego, Calif.)), and SOLiD® and Ion Torrent™ technologies (available from Life Technologies Corp. (Carlsbad, Calif.)).

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference in its entirety.

While the foregoing specification teaches the principles of the invention, with examples provided for the purpose of illustration, it will be appreciated by one skilled in the art from reading this disclosure that various changes in form and detail can be made without departing from the true scope of the invention.

### EXAMPLES

### Example 1

### Whole-Genome Association Study

A whole-genome association (WGA) study was undertaken in which the case group comprised 151 cases. Patients with inflammatory bowel disease who while taking one of the 5-aminosalicylate drugs (sulfasalazine, mesalazine, balsalazide, olsalazine) developed a 50% rise in their serum creatinine that their treating physician suspected was caused by the 5-ASA agent and subsequently stopped the drug were identified. Patients were required to have normal renal function prior to initiation of 5-ASA agents to be eligible for this study. An expert adjudication panel assessed causality for all cases to exclude confounders and other possible causes of renal injury. Standardized phenotypic definitions for 5-ASA nephrotoxicity are described in Heap, G A, et al. Clinical Features and HLA Association of 5-Aminosalicylate (F-ASA) Induced Nephrotoxicity in Inflammatory Bowel Disease (manuscript attached as Appendix A), the contents of which are incorporated by reference.

The control group comprised 4109 samples that matched for sex, disease status, and race.

Genotyping was performed using Illumina HumanCoreExome chips.

Principle component analysis (PCA) was done on all 5-ASA nephrotoxicity cases and controls to detect population structure. Standard quality control procedures were applied to the case-control genotype data set (based on SNP call rates, Hardy-Weinberg Equilibrium, and minor allele frequency) to exclude from downstream analysis low quality SNPs that could generate potentially false positive associations. Genetically-matched controls were selected for each case group, resulting in 143 cases.

Associations were tested using Fisher's exact test under additive, dominant, and recessive models through PLINK. The cohorts analyzed against the 4109 controls in the WGA study were all cases of 5-ASA nephrotoxicity and cases of 5-ASA nephrotoxicity that had undergone a renal biopsy with a confirmed diagnosis of interstitial nephritis (n=55 cases).

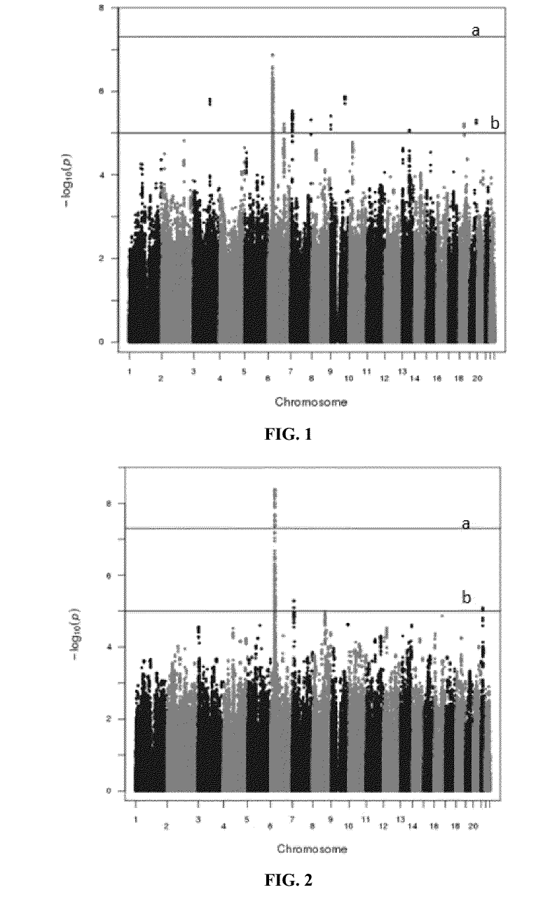

FIG. 1 is a Manhattan Plot that shows the association of each tested SNP with nephrotoxicity to 5-ASA agents for all samples tested. The line b represents P=1×10⁻⁵ and line a represents P=5×10⁻⁸. FIG. 2 is a Manhattan Plot that shows the association of each tested SNP with nephrotoxicity to 5-ASA agents when only those samples with a diagnosis of interstitial nephritis on renal biopsy are included to refine the phenotype definition.

Table 1 shows the SNPs that have a p-value smaller than 10⁻⁵ in the data set. Table 1 also shows the SNPs found to be the most strongly associated with 5-ASA nephrotoxicity.

TABLE 1

| | Allele | Allele | | | Odds | Standard | |
|---|---|---|---|---|---|---|---|
| SNP Name | 1 | 2 | Frequency | INFO | Ratio | Error | p-value |
| rs3135356 | T | C | 0.166 | 1.0439 | 3.1065 | 0.1931 | 4.35E−09 |
| rs3135351 | T | G | 0.166 | 1.0439 | 3.1065 | 0.1931 | 4.35E−09 |
| rs2395171 | T | C | 0.1679 | 1.0396 | 3.0797 | 0.1937 | 6.35E−09 |
| rs2395162 | T | G | 0.1687 | 1.0296 | 3.0813 | 0.1942 | 6.85E−09 |
| rs2395161 | C | A | 0.1686 | 1.0451 | 3.0467 | 0.1927 | 7.41E−09 |
| rs2001097 | A | C | 0.1684 | 1.0464 | 3.0435 | 0.1927 | 7.62E−09 |
| rs3135380 | G | T | 0.1684 | 1.0464 | 3.0435 | 0.1927 | 7.62E−09 |
| rs3135378 | T | C | 0.1684 | 1.0464 | 3.0435 | 0.1927 | 7.62E−09 |
| rs3135376 | C | T | 0.1684 | 1.0464 | 3.0435 | 0.1927 | 7.62E−09 |
| rs3135375 | T | C | 0.1684 | 1.0464 | 3.0435 | 0.1927 | 7.62E−09 |
| rs3135374 | T | G | 0.1684 | 1.0464 | 3.0435 | 0.1927 | 7.62E−09 |
| rs3135372 | T | G | 0.1684 | 1.0464 | 3.0435 | 0.1927 | 7.62E−09 |
| rs2187820 | A | G | 0.1684 | 1.0464 | 3.0435 | 0.1927 | 7.62E−09 |
| rs2213580 | G | A | 0.1684 | 1.0464 | 3.0435 | 0.1927 | 7.62E−09 |
| rs9268530 | C | T | 0.1684 | 1.0463 | 3.0435 | 0.1927 | 7.62E−09 |
| rs9268534 | G | T | 0.1684 | 1.0463 | 3.0435 | 0.1927 | 7.62E−09 |
| rs3135382 | C | A | 0.1684 | 1.0463 | 3.0435 | 0.1927 | 7.62E−09 |
| rs6930571 | T | G | 0.1684 | 1.0463 | 3.0435 | 0.1927 | 7.62E−09 |
| rs6908065 | C | A | 0.1678 | 1.0419 | 3.0547 | 0.1934 | 7.68E−09 |
| rs6930933 | T | G | 0.1686 | 1.0453 | 3.0432 | 0.1927 | 7.69E−09 |
| rs2395164 | T | C | 0.1685 | 1.0461 | 3.0422 | 0.1927 | 7.77E−09 |
| rs3129887 | A | G | 0.1737 | 1.0442 | 2.9457 | 0.1929 | 2.14E−08 |
| rs3135353 | A | G | 0.1447 | 1.0458 | 2.9886 | 0.1971 | 2.80E−08 |
| rs2105902 | A | T | 0.1447 | 1.0447 | 2.9897 | 0.1973 | 2.85E−08 |
| rs2239805 | C | A | 0.1696 | 1.0397 | 2.9361 | 0.1943 | 2.99E−08 |
| rs1051336 | A | G | 0.1716 | 1.0361 | 2.9084 | 0.1948 | 4.26E−08 |
| rs1041885 | T | A | 0.1716 | 1.0361 | 2.9084 | 0.1948 | 4.26E−08 |
| rs2239806 | A | G | 0.1716 | 1.0361 | 2.9083 | 0.1948 | 4.27E−08 |
| rs3129884 | C | T | 0.1716 | 1.0361 | 2.9083 | 0.1948 | 4.27E−08 |
| rs3135393 | C | T | 0.1708 | 1.0536 | 2.8595 | 0.1924 | 4.71E−08 |
| rs3129963 | G | A | 0.1806 | 1.039 | 2.8291 | 0.1933 | 7.49E−08 |
| AA_DRB1_26_32660070_Y | P | A | 0.1453 | 1.0327 | 2.773 | 0.2 | 3.40E−07 |
| SNP_DRB1_32660070 | T | A | 0.1453 | 1.0327 | 2.7727 | 0.2 | 3.41E−07 |
| AA_DRB1_37_32660037_N | P | A | 0.2385 | 1.0219 | 2.6362 | 0.1904 | 3.57E−07 |
| SNP_DRB1_32660038_T | P | A | 0.2385 | 1.0218 | 2.6361 | 0.1904 | 3.57E−07 |
| rs1794282 | A | G | 0.1133 | 1.0106 | 2.931 | 0.2128 | 4.35E−07 |
| rs3129716 | C | T | 0.134 | 1.0435 | 2.772 | 0.2021 | 4.51E−07 |
| rs2395158 | G | A | 0.1587 | 1.0415 | 2.708 | 0.1977 | 4.66E−07 |
| rs3129953 | T | C | 0.1589 | 1.0399 | 2.7096 | 0.1978 | 4.69E−07 |
| rs2854275 | T | G | 0.1345 | 1.0463 | 2.7535 | 0.2017 | 5.11E−07 |
| rs9273327 | C | A | 0.1345 | 1.0465 | 2.7529 | 0.2017 | 5.14E−07 |
| HLA_DRB1_0301 | P | A | 0.1347 | 1.0456 | 2.7515 | 0.2019 | 5.37E−07 |
| AA_DRB1_77_32659917 | N | T | 0.1348 | 1.0452 | 2.7514 | 0.2019 | 5.38E−07 |
| SNP_DRB1_32659917 | T | G | 0.1348 | 1.0452 | 2.7514 | 0.2019 | 5.38E−07 |
| AA_DRB1_74_32659926_R | P | A | 0.1348 | 1.0452 | 2.7514 | 0.2019 | 5.38E−07 |
| SNP_DRB1_32659926_C | P | A | 0.1348 | 1.0451 | 2.7513 | 0.2019 | 5.39E−07 |
| HLA_DRB1_03 | P | A | 0.1348 | 1.0451 | 2.7512 | 0.2019 | 5.40E−07 |
| SNP_DRB1_32657541 | A | G | 0.1349 | 1.0452 | 2.7498 | 0.202 | 5.49E−07 |
| rs3129843 | G | A | 0.1174 | 1.0437 | 2.8393 | 0.2084 | 5.53E−07 |
| rs1059615 | T | C | 0.135 | 1.0445 | 2.7488 | 0.202 | 5.56E−07 |
| rs3129950 | C | G | 0.1177 | 1.0423 | 2.8348 | 0.2086 | 5.86E−07 |
| rs9268219 | G | T | 0.1102 | 0.9569 | 3.0801 | 0.2253 | 5.94E−07 |
| rs2856674 | C | T | 0.1353 | 1.0448 | 2.7398 | 0.2019 | 5.99E−07 |
| rs2187668 | A | G | 0.1356 | 1.0398 | 2.7396 | 0.2019 | 6.00E−07 |
| rs2284189 | C | T | 0.1356 | 1.0398 | 2.7396 | 0.2019 | 6.00E−07 |
| rs3129927 | C | A | 0.1185 | 1.038 | 2.8315 | 0.2093 | 6.59E−07 |
| rs3135394 | C | T | 0.1189 | 1.0294 | 2.8269 | 0.2091 | 6.68E−07 |
| rs9268235 | T | C | 0.1185 | 1.0392 | 2.8293 | 0.2093 | 6.69E−07 |
| rs9268177 | A | C | 0.1185 | 1.0391 | 2.8293 | 0.2093 | 6.73E−07 |
| rs7775397 | G | T | 0.1186 | 1.0393 | 2.8275 | 0.2093 | 6.82E−07 |
| rs3132971 | G | T | 0.1186 | 1.0412 | 2.8199 | 0.209 | 7.07E−07 |
| rs2395149 | A | G | 0.1187 | 1.0394 | 2.8194 | 0.209 | 7.08E−07 |
| HLA_DQB1_0201 | P | A | 0.137 | 1.0354 | 2.723 | 0.2021 | 7.20E−07 |
| rs3129939 | G | A | 0.1877 | 1.0444 | 2.5899 | 0.1934 | 8.68E−07 |
| rs2143461 | T | C | 0.1878 | 1.0434 | 2.5899 | 0.1936 | 8.81E−07 |
| rs3129924 | T | C | 0.1881 | 1.0432 | 2.5837 | 0.1935 | 9.27E−07 |
| rs3129925 | A | G | 0.1881 | 1.0432 | 2.5837 | 0.1935 | 9.27E−07 |
| rs3129926 | C | T | 0.1881 | 1.0432 | 2.5837 | 0.1935 | 9.27E−07 |
| rs1265757 | A | G | 0.1202 | 1.0343 | 2.7971 | 0.2097 | 9.33E−07 |
| rs1265754 | T | A | 0.1202 | 1.0343 | 2.7971 | 0.2097 | 9.33E−07 |
| rs910051 | C | A | 0.1208 | 1.035 | 2.7843 | 0.2098 | 1.06E−06 |
| rs3864299 | T | A | 0.2241 | 1.03 | 2.5426 | 0.1919 | 1.15E−06 |
| rs2143462 | T | C | 0.1897 | 1.0423 | 2.5597 | 0.1935 | 1.19E−06 |
| rs6909790 | G | A | 0.2244 | 1.0323 | 2.5351 | 0.1915 | 1.19E−06 |
| rs9268213 | G | A | 0.2244 | 1.0324 | 2.5348 | 0.1915 | 1.20E−06 |
| rs9268215 | G | A | 0.2244 | 1.0324 | 2.5347 | 0.1915 | 1.20E−06 |
| rs6915455 | A | G | 0.2245 | 1.032 | 2.5341 | 0.1916 | 1.21E−06 |
| rs7742654 | G | C | 0.2245 | 1.0322 | 2.5338 | 0.1916 | 1.22E−06 |
| rs3864300 | T | G | 0.2245 | 1.0322 | 2.5338 | 0.1916 | 1.22E−06 |
| rs9268165 | G | A | 0.2245 | 1.0322 | 2.5338 | 0.1916 | 1.22E−06 |
| rs9268166 | C | T | 0.2245 | 1.0322 | 2.5338 | 0.1916 | 1.22E−06 |
| rs6934429 | T | G | 0.2245 | 1.0316 | 2.5347 | 0.1917 | 1.22E−06 |
| rs1018434 | G | A | 0.2245 | 1.0307 | 2.5352 | 0.1917 | 1.22E−06 |
| rs9268192 | T | C | 0.2245 | 1.032 | 2.5336 | 0.1916 | 1.22E−06 |
| rs1018433 | A | T | 0.2245 | 1.032 | 2.5337 | 0.1916 | 1.22E−06 |
| rs6457536 | G | A | 0.2245 | 1.0321 | 2.5336 | 0.1916 | 1.22E−06 |
| rs9268198 | T | C | 0.2245 | 1.032 | 2.5334 | 0.1916 | 1.22E−06 |
| rs7341328 | A | G | 0.2246 | 1.0316 | 2.5332 | 0.1916 | 1.23E−06 |
| rs9276731 | T | G | 0.1099 | 1.0397 | 2.7995 | 0.2128 | 1.32E−06 |
| rs7762279 | C | T | 0.1102 | 1.0372 | 2.796 | 0.213 | 1.39E−06 |
| rs3129891 | A | G | 0.1961 | 1.0156 | 2.5284 | 0.1968 | 2.43E−06 |
| SNP_DRB1_32659926_CA | P | A | 0.162 | 1.0355 | 2.552 | 0.1989 | 2.47E−06 |
| AA_DRB1_74_32659926_RL | P | A | 0.162 | 1.0355 | 2.5519 | 0.1989 | 2.47E−06 |
| AA_DRB1_74_32659926_RE | P | A | 0.1822 | 0.9817 | 2.5609 | 0.201 | 2.89E−06 |
| rs3131643 | T | C | 0.1578 | 1.0202 | 2.5693 | 0.2028 | 3.27E−06 |
| rs9275602 | A | C | 0.1808 | 0.883 | 2.6826 | 0.2123 | 3.36E−06 |
| rs3135363 | C | T | 0.2538 | 1.0357 | 2.3974 | 0.1895 | 3.93E−06 |
| rs3129933 | A | G | 0.1847 | 1.0373 | 2.4655 | 0.1962 | 4.22E−06 |
| rs7745174 | A | G | 0.1849 | 1.0347 | 2.4661 | 0.1962 | 4.24E−06 |
| rs9268220 | T | C | 0.1849 | 1.0347 | 2.4661 | 0.1963 | 4.24E−06 |
| rs9268125 | C | T | 0.2224 | 1.0287 | 2.4253 | 0.1927 | 4.26E−06 |
| rs6910668 | G | T | 0.1853 | 1.0343 | 2.4595 | 0.1962 | 4.51E−06 |
| rs9268127 | C | T | 0.1854 | 1.0366 | 2.4518 | 0.1959 | 4.71E−06 |
| rs7750783 | T | C | 0.225 | 1.0156 | 2.4222 | 0.1937 | 4.97E−06 |
| rs1033499 | T | C | 0.1866 | 1.0327 | 2.4459 | 0.1966 | 5.41E−06 |
| SNP_DRB1_32660038_A | P | A | 0.7421 | 1.0259 | 0.4215 | 0.19 | 5.43E−06 |
| rs1559873 | C | T | 0.2217 | 1.03 | 2.4023 | 0.1929 | 5.51E−06 |
| AA_DRB1_37_32660037_NL | P | A | 0.2581 | 1.025 | 2.3716 | 0.1901 | 5.53E−06 |
| rs9268212 | C | T | 0.2216 | 1.0272 | 2.4047 | 0.1932 | 5.60E−06 |
| rs9268197 | A | C | 0.2216 | 1.0272 | 2.4046 | 0.1932 | 5.60E−06 |
| rs9268176 | T | C | 0.2216 | 1.0272 | 2.4045 | 0.1932 | 5.61E−06 |
| rs3115560 | T | C | 0.2347 | 1.0405 | 2.3745 | 0.1905 | 5.62E−06 |
| rs3132928 | A | G | 0.2348 | 1.0388 | 2.3767 | 0.1907 | 5.62E−06 |
| rs6939410 | G | A | 0.2216 | 1.0272 | 2.4042 | 0.1932 | 5.63E−06 |
| rs9268202 | T | C | 0.2216 | 1.0272 | 2.4042 | 0.1932 | 5.63E−06 |
| rs6934776 | A | G | 0.2216 | 1.0272 | 2.4042 | 0.1932 | 5.63E−06 |
| rs3132931 | G | T | 0.2215 | 1.0292 | 2.4017 | 0.193 | 5.63E−06 |
| rs3096674 | A | G | 0.2215 | 1.0291 | 2.4012 | 0.193 | 5.67E−06 |
| rs7775332 | T | A | 0.235 | 1.0389 | 2.3742 | 0.1906 | 5.75E−06 |
| rs1018430 | A | G | 0.235 | 1.0395 | 2.3728 | 0.1906 | 5.78E−06 |
| rs3096677 | C | A | 0.2217 | 1.0299 | 2.3975 | 0.1929 | 5.81E−06 |
| rs6909427 | G | T | 0.2351 | 1.0394 | 2.3723 | 0.1906 | 5.82E−06 |
| rs9268167 | A | C | 0.235 | 1.0395 | 2.3723 | 0.1906 | 5.82E−06 |
| rs3115557 | A | G | 0.2217 | 1.03 | 2.3969 | 0.1929 | 5.85E−06 |
| rs3096681 | C | T | 0.235 | 1.0413 | 2.3694 | 0.1904 | 5.87E−06 |
| rs3132945 | C | T | 0.235 | 1.0411 | 2.3689 | 0.1904 | 5.90E−06 |
| rs3096673 | C | T | 0.2351 | 1.041 | 2.369 | 0.1904 | 5.91E−06 |
| rs9268055 | C | T | 0.2351 | 1.0405 | 2.3687 | 0.1905 | 5.96E−06 |
| rs3115563 | T | A | 0.2351 | 1.0405 | 2.3687 | 0.1905 | 5.96E−06 |
| rs3749966 | C | T | 0.2351 | 1.0408 | 2.3684 | 0.1904 | 5.97E−06 |
| rs9268135 | G | A | 0.2218 | 1.0301 | 2.3944 | 0.1929 | 5.98E−06 |
| rs9268131 | G | A | 0.2219 | 1.0303 | 2.3935 | 0.1928 | 6.01E−06 |
| rs3130340 | C | T | 0.2351 | 1.0417 | 2.3659 | 0.1903 | 6.03E−06 |
| rs2050189 | G | A | 0.217 | 0.5679 | 3.0712 | 0.248 | 6.04E−06 |
| rs3864302 | A | G | 0.2353 | 1.0401 | 2.3676 | 0.1905 | 6.05E−06 |
| rs6935269 | C | T | 0.2353 | 1.041 | 2.3665 | 0.1904 | 6.08E−06 |
| rs9268137 | A | G | 0.222 | 1.029 | 2.3934 | 0.1929 | 6.08E−06 |
| rs3115553 | A | G | 0.2353 | 1.0424 | 2.362 | 0.1902 | 6.21E−06 |
| rs7751896 | A | G | 0.2354 | 1.0419 | 2.3629 | 0.1903 | 6.23E−06 |
| rs3115569 | T | C | 0.2355 | 1.0404 | 2.3639 | 0.1905 | 6.27E−06 |
| rs3117103 | A | T | 0.1298 | 1.0384 | 2.5586 | 0.209 | 6.98E−06 |
| rs1559874 | A | G | 0.2231 | 1.0317 | 2.3729 | 0.1927 | 7.29E−06 |
| AA_DRB1_74_32659926_AQ | P | A | 0.7908 | 0.9789 | 0.4108 | 0.1988 | 7.63E−06 |
| rs9276601 | G | A | 0.1704 | 1.031 | 2.4243 | 0.2002 | 9.73E−06 |

### REFERENCES

Sambrook et al., Molecular Cloning, Cold Spring Harbor Laboratory Press, Cold Spring Harbor, N.Y., 1989.
Innis et al., Proc. Natl. Acad. Sci. USA, 85(24): 9436-9449, 1988.
Guilfoyle et al., Nucleic Acids Research, 25: 1854-1858, 1997.
Walker et al., Proc. Natl. Acad. Sci. USA, 89: 392-396, 1992.
Kwoh et al., Proc. Natl. Acad. Sci. USA, 86: 1173, 1989.
Frohman, PCR Protocols: A Guide to Methods and Applications, Academic Press, N.Y., 1990.
Ohara et al., Proc. Natl. Acad. Sci. USA, 86: 5673-5677, 1989.

### Example 2

### Clinical Features and HLA Association of 5-Aminosalicylate (5-ASA) Induced Nephrotoxicity in Inflammatory Bowel Disease

Background & Aims: Nephrotoxicity is a rare idiosyncratic reaction to 5-aminosalicylate (5-ASA) therapies. The aims of this study were to describe the clinical features of this complication and identify clinically useful genetic markers so these drugs can be avoided, or monitoring intensified, in high-risk patients.

Methods: Inflammatory bowel disease patients were recruited from 89 sites around the world. Inclusion criteria included normal renal function prior to commencing 5-ASA, ≧50% rise in creatinine any time after starting 5-ASA and physician opinion implicating 5-ASA strong enough to justify drug withdrawal. An adjudication panel identified definite and probable cases from structured case report forms. A genome wide association study was then undertaken with these cases and 4,109 disease controls.

Results: After adjudication, 151 cases of 5-ASA induced nephrotoxicity were identified. 68% of cases were males, with nephrotoxicity occurring at a median age of 39.4 years (range 6-79 years). The median time for development of renal injury after commencing 5-ASA was 3.0 years (95% CI 2.3-3.7). Only 30% of cases recovered completely after drug withdrawal with 15 patients requiring permanent renal replacement therapy. A genome-wide association study identified a suggestive association in the HLA region (P=1×10⁻⁷) with 5-ASA induced nephrotoxicity. A sub-group analysis of patients who had a renal biopsy demonstrating interstitial nephritis (n=55) significantly strengthened this association (P=4×10⁻⁹, Odds Ratio 3.1).

Conclusions: This is the largest and most detailed study of 5-ASA induced nephrotoxicity to date. It highlights the morbidity associated with this condition and identifies for the first time a significant genetic predisposition to a drug induced renal injury.

Introduction

5-aminosalicylate (5-ASA) medications are the most frequently prescribed class of drug to induce and maintain remission in patients with mild-to-moderately active ulcerative colitis¹. Originally administered in combination with sulfapyridine as sulfasalazine, 5-ASA is now more often coated with a resin/gel or as a pro-drug/dimer to enhance distal bowel delivery through preparations such as mesalazine, olsalazine and balsalazide². The use of these agents in maintenance therapy over decades means that long-term toxicity is an important consideration.

Nephrotoxicity associated with 5-ASA agents was first described in animal models and case reports in the 1970s and has since been reported multiple times to both sulfasalazine and the more modern 5-ASA agents³⁻⁸. In 1990 the UK Committee on Safety of Medicines issued an alert on nephrotoxic reactions to mesalazine⁹. Data from clinical trials suggest an annual risk of 0.26% and data from a questionnaire sent to gastroenterologists estimated an incidence of 1 case per 4000 patient years^{10,11}. A review of the UK General Practice Research Database calculated the incidence at 0.17 cases per 100 patients per year but the authors noted that only 13% of these patients had a histological diagnosis of interstitial nephritis¹². Regular monitoring of renal function for the duration of therapy is recommended, although the cost-effectiveness of this approach has not been demonstrated.

Rare idiosyncratic drug reactions are often notoriously difficult to characterise due to the small number of cases available to individual researchers. The International Serious Adverse Events Consortium was launched in 2007 to facilitate the collection of large cohorts of patients who developed these rare serious drug side effects¹³. Members of this consortium have recently demonstrated the utility of using small numbers of well-characterised cases to identify strong, clinically useful genetic risk factors for serious adverse drug reactions through genome-wide association study methodologies. Good examples of this approach are the identification of HLA-B*57:01 as a major determinant of cholestatic liver injury associated with flucloxacillin and our recent identification of an association between HLA-DRB1*07:01 and thiopurine induced pancreatitis^{14,15}.

Described herein, for the first time, are a cohort of patients with inflammatory bowel disease (IBD) who developed nephrotoxicity subsequent to 5-ASA administration. This cohort was used to characterize the clinical features of this serious adverse event and then perform the first genome wide association study to identify genetic risk factors for the development of a drug-induced renal injury.

Methods

Patient Recruitment: Individual study sites identified and recruited patients with 5-ASA induced nephrotoxicity. This study was open to recruitment at 118 UK research sites (73.8% of the 160 acute NHS trusts in the UK) as well as 45 international sites. In total 77 sites from the UK and 12 sites from outside the UK recruited one or more patients. The protocol was approved by the National Research Ethics Committee South West, Exeter, UK (10/H0203/76) and by all local research and development offices.

Inclusion criteria for patient recruitment required the presence of all of the following:
Patient administered any 5-ASA compound for treatment of inflammatory bowel disease (Crohn's disease, ulcerative colitis or IBD-unclassified)
Patient aged 6 or over
Normal creatinine or estimated glomerular filtration rate (eGFR) prior to first administration of 5-ASA or a creatinine that returned to the normal range after cessation of therapy
Greater than or equal to 50% rise in serum creatinine any time after introduction of 5-ASA
Physician opinion implicating 5-ASA strong enough to justify drug withdrawal, even if temporary

Cases were identified from recruiting sites through clinics and systematic searches of historical records and pathology databases. Gastroenterologists who replied to a 2001-2002 UK survey of 5-ASA induced nephrotoxicity were encouraged to submit cases¹⁰. Clinicians who had submitted adverse drug reaction reports to the Medicines and Healthcare Products Regulatory Agency (MHRA) were invited to consider recruiting patients. Direct advertising to patients through the national patient newsletter was also undertaken. Cases were recruited who developed nephrotoxicity between 1988 and 2013 (73% of cases were diagnosed with renal injury after the year 2000).

Case adjudication: An anonymised case report form detailing demographic, clinical and drug history was completed with the aid of hospital records. Two 6 ml EDTA blood samples were taken at this visit for DNA extraction (BD Vacutainer, USA). The case report forms also requested creatinine levels and their corresponding dates at four time points: 1) at baseline (usually before 5-ASA commenced, but not exclusively), 2) at the recording of first abnormal creatinine value, 3) the worst creatinine value and 4) the best recovered creatinine value. After data collection, the last normal creatinine value before development of renal injury was also obtained, if it was available (92/151 cases), to enable better characterisation of the time period. If a renal biopsy was performed, the anonymised report was requested.

To assess patient eligibility for entry to this study, at least three gastroenterologists and at least one nephrologist reviewed each case for causality at a dedicated in-person adjudication panel meeting. For each case the evidence implicating the 5-ASA as the cause of nephrotoxicity was assessed using an adapted version of the validated Liverpool Adverse Drug Reaction Causality Assessment Tool¹⁶. Patients were classified as definite, probable, possible or unlikely cases of 5-ASA nephrotoxicity based on the adjudicator's independent assessment that the nephrotoxicity was due to 5-ASA treatment. The panel discussed all cases before a final adjudication decision was reached. Only individuals classified as probable or definite cases of 5-ASA induced nephrotoxicity were taken forward for clinical and genetic analyses.

Concomitant administration of any medications known to cause nephrotoxicity classified the patient as a possible case and these cases were excluded. This included the use of a) Antibiotics: penicillins, cephalosporins, ciprofloxacin, sulphonamides, rifampicin, b) Diuretics: furosemide, bumetanide, thiazides, c) Non-steroidal anti-inflammatory drugs, d) Proton pump inhibitors, e) allopurinol, f) cimetidine, g) indinivir. The presence of uncontrolled diabetes, uncontrolled hypertension or peripheral vascular disease also classified the patient as a possible case of 5-ASA induced nephrotoxicity and they were not taken forward for analysis. A patient treated with 5-ASA for microscopic colitis was recruited in error but was excluded during the adjudication process.

Definite cases required the development of renal injury upon rechallenge with 5-ASA. Cases classified as probable demonstrated a temporal relationship with 5-ASA administration with no other identifiable risk factors for renal injury as described above.

DNA Extraction and Genotyping: DNA was extracted from EDTA stabilised blood using the Qiagen Autopure LS with Puregene chemistry. Samples were genotyped on the Illumina Infinium HumanCoreExome beadchip (Illumina, USA), which contains 264,909 haplotype tagging SNP markers, and 244,593 exome focused markers by the Broad Institute (Boston, USA).

Clinical data analysis: Paper case report forms were entered into the electronic database before independent, two-person, un-blinded data cleaning was undertaken to ensure data quality prior to analysis. All data was analysed in R 3.0.2. Listwise deletion was used for missing data. Normality was tested for by the Shapiro-Wilks normality test (P<0.05 considered non-parametric). Non-parametric data is presented with median values and the 95% confidence intervals of the median with comparisons performed by a Wilcoxon signed rank test or chi-squared test as appropriate. Logistic and multivariate regression was conducted in R with pre-set variables (no stepwise regression). The definition of a return to baseline creatinine was a return to the upper limit of the local hospital labs normal range or 120 μmol/l if this range was not known.

Genome-wide and HLA Imputation and association analyses: Three samples failed DNA extraction and were unavailable for genetic analysis. Genotyping was performed on the remaining 148 cases adjudicated as definite or probable 5-ASA induced nephrotoxicity using the HumanCoreExome SNP Chip. Genotypes were called using Gencall¹⁷. We excluded SNPs with a HWE P<0.0001 and a genotype success rate<0.99. We excluded indels. Exclusion criteria for case samples were genotyping success rate<0.98 and a heterozygosity rate>4SD (no samples were removed based on these criteria). To improve calling of low-frequency variants we used zCall¹⁸. After running zCall, SNPs were excluded if they had a HWE P<0.0001, MAF<0.01 or if they were duplicated. This left 264,088 autosomal SNPs for imputation. The control patients with Crohn's disease and ulcerative colitis were obtained from the UK IBD Genetics Consortium as part of the Wellcome Trust Case Control Consortium (WTCCC 1 for Crohn's disease and WTCCC 2 for Ulcerative Colitis)^{19,20}. There were 1748 CD control samples genotyped on the Affymetrix 500K SNP chip and 2361 UC samples genotyped on the Affymetrix 6 SNP chip available for this analysis. Preliminary QC had already been performed on the 1748 CD and 2361 UC samples^{19,20}. From these two control cohorts, SNPs with a genotyping success rate<0.99, MAF<0.01 and a HWE P<0.0001 were excluded. This left 396,255 (CD) and 727,195 (UC) autosomal SNPs. To exclude ethnic outliers principal components analysis was performed using GCTA²¹. To generate the principal components d a set of 36,702 SNPs were used that were imputed with R²>0.99 in the cases (see below) and directly genotyped in the two control cohorts and were not in strong linkage (r²<0.2).

Four 5-ASA nephrotoxicity cases and 62 control samples were excluded for being>4SD from the first or second principal components. KING²² was used to test for cryptic relatedness between samples. If a case and control pair of samples had a kinship coefficient>0.2 we excluded the control sample, otherwise one of the pair of samples was excluded at random. One case and 13 control samples were excluded because of relatedness to other case or control samples. After exclusions this left 143 “probable” and “definite cases”. The ratio of Crohn's disease to ulcerative colitis patients in the control group (59%) was similar to that in the case cohort (60%).

Genome-wide and HLA Imputation and association analyses: As previously¹⁵, minimac²³ was used to impute into the European phase1 version 3 (20101123) SNP and indels reference panel to prevent spurious associations due to variations in genotyping chips between cohorts. 76% of the 9,412,474 SNPs with MAF>1% frequency were imputed at R²>0.6 in the cases; 75% in the CD controls and 82% in the UC controls. As each of the three case and control cohorts used a different SNP genotyping chip subsequent association analyses were focused on a very conservative subset of 2,883,071 SNPs that had an imputation R²>0.95 in all three cohorts. For dedicated imputation of the HLA region we used SNP2HLA²⁴ and imputed into the T1DGC reference panel of 5,224 individuals that have had classical HLA alleles typed as well as SNPs and indels by the immunochip. 8398 of the 8961 variants in the T1DGC panel were captured with an INFO score>0.8. Mach2dat²⁵ was used to perform association analyses for the genome-wide analyses, and PLINK²⁶ was used to perform association analyses for the HLA imputed analyses.

Data access: Phenotype and genotype data for cases is freely available upon request from the iSAEC Data Access Committee for users who comply with the Consortium's Data Release and IP Policy¹³. Data will be available from https://dataportal.saeconsortium.org/ within 12 months of genotype completion. Raw genotype data is freely available to researchers upon request. For further data access details please contact: saec@c2b2.columbia.edu.

Genotype data for the WTCCC Ulcerative colitis and Crohn's disease cases are available from the European Genome-Phenome Archive at https://www.ebi.ac.uk/ega/home.

Results

Patient identification and adjudication: Through our international network of research sites a total of 204 IBD patients with suspected 5-ASA-induced nephrotoxicity were recruited. All cases underwent a rigorous assessment of causality at an adjudication panel composed of nephrologists and gastroenterologists using a validated tool¹⁶. After this panel meeting the development of nephrotoxicity could be confidently assigned to the administration of 5-ASA medications for 151 out of 204 patients. Of these 151 cases, 5 were classified as definite cases of 5-ASA induced nephrotoxicity, as they had a second episode of kidney injury when re-challenged with the agent. The remaining 146 cases were classified as probable cases.

Clinical features of 5-ASA induced nephrotoxicity: The 151 patients who were adjudicated as definite or probable cases comprised 58 patients with Crohn's disease, 88 patients with ulcerative colitis and 5 patients with IBD unclassified. 68% of cases recruited were male. The median age at diagnosis of Crohn's disease was 29.5 years (95% CI 25.2-33.9) while the median age for ulcerative colitis was 29.7 years (26.7-32.8). 146 patients (97%) self identified as being of white ethnicity, 2 patients did not provide a reply while 3 patients reported mixed ancestry. A summary of the disease activity and location in the two years prior to development of renal disease is show in Table 2.

The median duration of 5-ASA treatment prior to first detection of a raised creatinine was 3.0 years (95% CI 2.3-3.7). 13% of cases reported an abnormal creatinine within the first 12 months of treatment. For 27% of patients we were unable to find a creatinine between the initiation of the drug and the first abnormal creatinine measurement. In these patients the median time from starting the drug to detection of nephrotoxicity was 4 years (95% CI 2.73-5.28) while the cohort with interval measurements had a median time of 2.5 years (95% CI 1.70-3.36, P=6×10⁻⁴). There was no significant difference in creatinine level at detection of abnormal renal function (P=0.75) or in the rate of recovery (P=0.72) between these two groups.

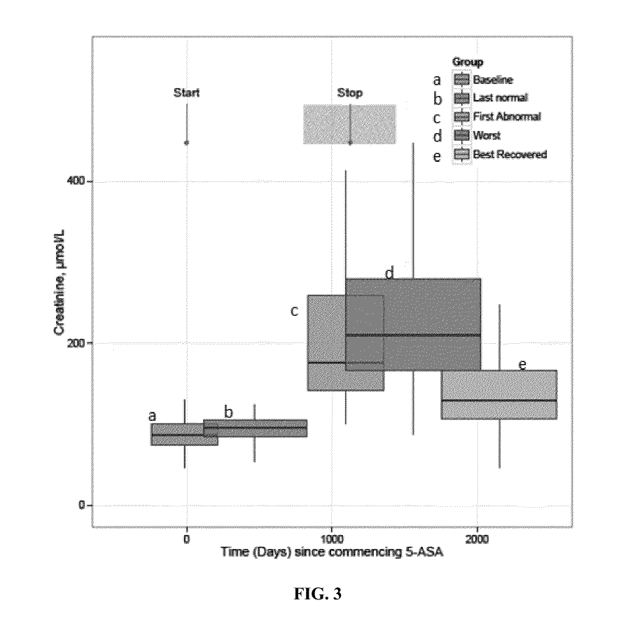

The majority of patients (91%) received oral 5-ASA alone with an average daily dose of 2.3 g (95% CI 2.1-2.5 g). 12 patients received a mix of oral and rectal 5-ASA, while 1 patient received only rectal 5-ASA preparations. The majority of patients were treated with mesalazine, however, most available 5-ASA preparations are represented in the data. FIG. 3 demonstrates the median creatinine levels and the time period at which these levels were collected at each time point for all 151 patients who developed nephrotoxicity.

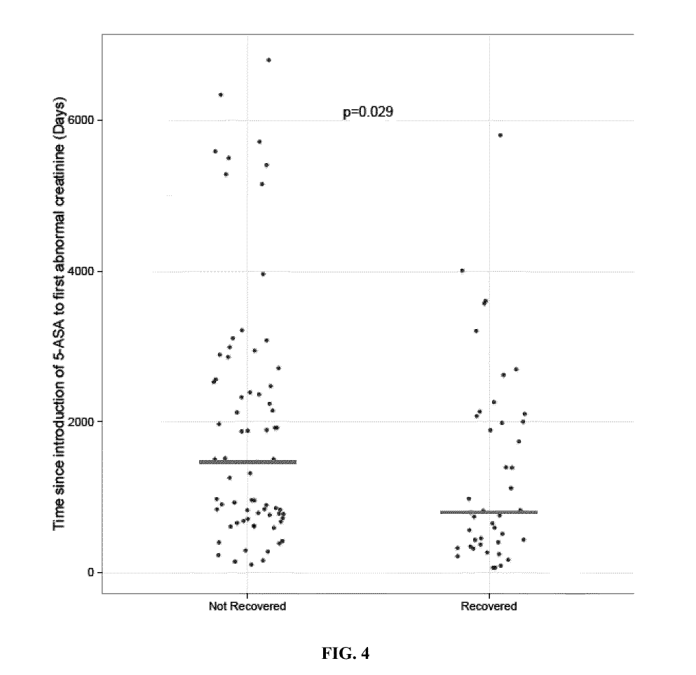

45 patients (30%) demonstrated full recovery of renal function within the follow-up period (median follow-up period 5.10 years 95% CI 4.17-6.02). A multivariate regression analysis was undertaken to investigate if any clinical features were predictive of renal function recovery after 5-ASA cessation. This analysis suggested that the length of 5-ASA treatment (P=0.05) and the average dose of 5-ASA (P=0.03) inversely correlated with the likelihood of renal function recovery suggesting cumulative toxicity. It has been suggested that patients who develop nephrotoxicity and stop the agent within 10 months of starting are more likely to recover to a normal creatinine²⁷. We were unable to replicate this association in our data (P=0.53), however, patients who did recover renal function appeared to have been taking 5-ASA for a shorter period of time before developing nephrotoxicity (Median 794 days, 95% CI 459.13-1128.87 vs. Median 1461 days, 95% CI 1008.80-1597.20, P=0.02, FIG. 4). 43% of patients were treated with steroids, which was not associated with a shorter time to recovery (P=0.20) or an increased rate of recovery (P=0.10).

15 out of 151 patients (9.9%) received renal replacement therapy, which for 13 patients took the form of a renal transplant. The remaining two patients were dialysis dependent at the date of study end. A multivariate regression analysis identified peak creatinine (P=0.008), treatment with steroids (P=0.037) and presence of a renal biopsy (P=0.045) as predictive of the need for renal replacement therapy.

In total 76 patients adjudicated as definite or probable underwent a renal biopsy, 57 of which demonstrated clear evidence of interstitial nephritis. Of these 58% demonstrated only chronic inflammatory changes in the interstitium, with 22% and 20% demonstrating acute or acute on chronic interstitial inflammation respectively. The remaining 19 biopsies demonstrated evidence of glomerulosclerosis or were non-diagnostic.

Genetic determinants of 5-Aminosalicylate (5-ASA) induced nephrotoxicity: The idiosyncratic nature of 5-ASA induced nephrotoxicity suggests that there may be a genetic basis for its development. To investigate this a genome wide association study was undertaken with the 151 cases described above and a control cohort of 1748 Crohn's and 2361 ulcerative colitis cases.

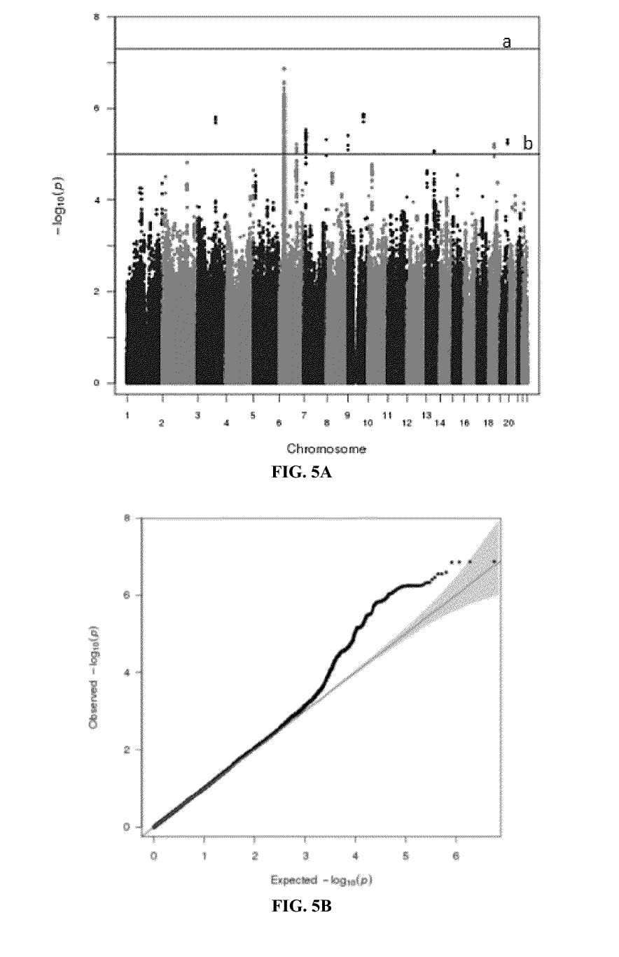

The strongest association signal for the development of nephrotoxicity was in the HLA region (rs3135349, Odds ratio 2.04, P=1×10⁻⁷) (see FIG. 5 for Manhattan and QQ plots). Therefore dedicated HLA imputation was performed using SNP2HLA in to the T1DGC reference panel. The top SNP after imputation was rs3135356, (OR=2.0, 95% CI 1.55-3.10, P=1×10⁻⁷. The results are shown in Table 3. This association was present in patients with Crohn's disease and ulcerative colitis when tested independently (P=1×10⁻⁷ and P=3×10⁻⁷).

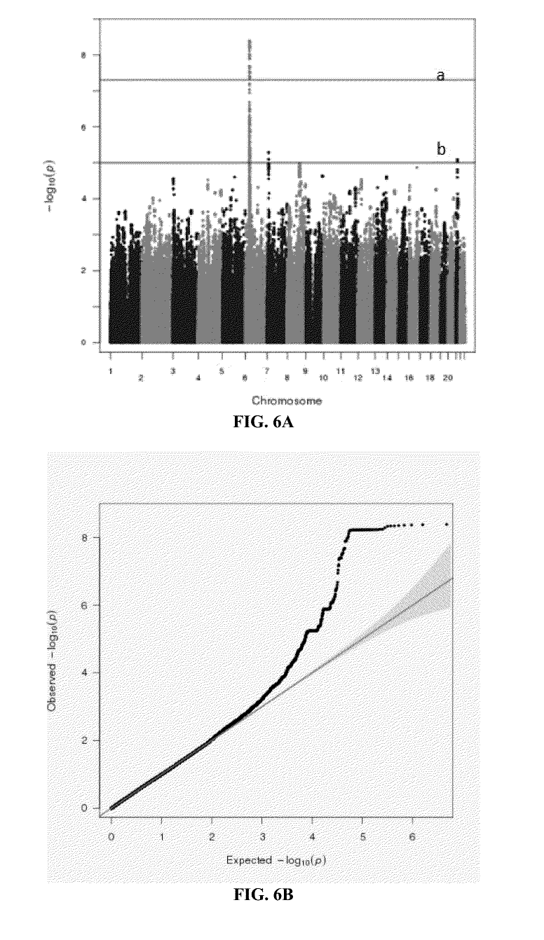

Despite the strict adjudication methods undertaken to assign causality of nephrotoxicity to the administration of 5-ASA compounds there might be other factors that have not been captured in the data collection that could impact on renal function. To further refine the phenotype definition therefore only those samples classified as definite or probable were studied, who also had a renal biopsy demonstrating interstitial nephritis (passed genetic QC n=55). Limiting the association analyses to the biopsy positive cases significantly strengthened the HLA association signal, despite the smaller number of cases, with the most associated SNP remaining rs3135356 (FIG. 6), but with an odds ratio 3.1, and a genome-wide significant P-value (P=4×10⁻⁹). This was robust to correction for the first 20 principle components OR=3.13, P=1×10⁻⁸. The most associated HLA allele from this analysis was HLA-DRB1*03:01 (P=5×10⁻⁷, Odds Ratio 2.76). This variant was not associated with duration of therapy prior to development of nephrotoxicity or the likelihood of recovery (P=0.63 & P=0.22).

Discussion

Presented herein is an analysis of the clinical features of patients with IBD who developed nephrotoxicity after administration of 5-ASA compounds. We have conducted the first ever genome wide association study of drug-induced renal injury and gone on to identify a marker within the HLA region associated with 5-ASA induced nephrotoxicity. The data from our cohort suggests that 5-ASA induced nephrotoxicity may present at any age and is more common in male patients. The histological hallmark is a chronic tubulointerstitial nephritis. Renal injury was detected after a median treatment time of 3 years following which only 30% of our cohort fully recovered renal function. In 10% of our cases, 5-ASA induced nephrotoxicity necessitated permanent renal replacement therapy.

Many drugs have been implicated in the development of interstitial nephritis, but proving causality is difficult. 5-ASA induced nephrotoxicity has been reported previously in case reports including a case of with a positive re-challenge²⁸. These reports, combined with the 151 cases here (including the five definite cases) provide compelling evidence that 5-ASA is able to cause renal injury and should be suspected in any patient with deteriorating renal function on these agents.

The temporal association between the use of 5-ASA and development of renal injury, the improvement on drug withdrawal (although this only occurs in 30% of patients) and the five patients who were re-challenged with 5-ASA with subsequent worsening of renal function provide evidence that the renal damage is likely to be drug related. The relationship between an increased likelihood of recovery and drug dose and duration also suggests a pathogenic role for the 5-ASA agents in interstitial nephritis development. However there has been a suggestion that the nephrotoxicity observed in IBD patients might be an extra-intestinal manifestation of disease rather than a result of drug toxicity^{12,31}. Four of the patients in this study had evidence of granulomatous interstitial nephritis with non-caseating granulomas seen on biopsy (1 patient with Crohn's disease, 2 with ulcerative colitis and 1 with IBD unclassified). This rare form of interstitial nephritis is most commonly seen in acute drug reactions but there are isolated case reports of patients with IBD developing interstitial nephritis with or without granulomas, some of whom have not been exposed to 5-ASAs³²⁻³⁴.

Current British Society of Gastroenterology guidelines (2011) recommend monitoring of renal function annually in patients taking 5-ASA agents, the European Crohn's and Colitis Organisation (2012) recommends monitoring in high risk patients while the American Gastroenterology Society (2010) recommends periodic monitoring noting evidence for a defined frequency is lacking³⁵⁻³⁷. The utility of these approaches has not been demonstrated, however, it has been noted that many patients do not have regular renal function monitoring whilst using 5-ASA³⁸. Indeed data from this study suggests that the median time from the last normal creatinine to the first abnormal value, which represents how often a patient is having a blood test, is 1.98 years with a range of 2 days to 15.3 years.

5-ASA agents are normally tolerated by the majority of patients suggesting an underlying genetic or environmental pre-disposition to the development of renal injury in a small subset of patients. The type of renal injury seen with 5-ASA appears to be consistent with the changes occasionally seen with long-term lithium use³⁹. Lithium ingestion over a prolonged period of time (usually greater than 2 years) has rarely been associated with the development of a chronic focal interstitial cortical fibrosis with mononuclear cell infiltrate—a chronic interstitial nephritis⁴⁰. Analogous to the renal injury seen with 5-ASA this typically occurs after a prolonged period of drug exposure and once identified by routine blood testing often fails to improve even after drug withdrawal⁴¹.

We have not attempted to replicate the association of rs3135356 in an independent population. The collection of cases described here required a collection period of 2 years and the involvement of 89 centres, and as such, further sample collection was felt to be unfeasible. An association between another HLA class II allele, HLA-DRB1*01:02 and the rare syndrome of tubulointerstitial nephritis and uveitis (TINU) has been described in the literature⁴². The association was only seen in patients with this syndrome and not in control patients with interstitial nephritis alone. This association is clearly distinct from the drug induced renal injury displayed here and is likely to reflect their separate aetiologies.

Carriage of the risk allele is associated with a 3 fold increased risk of renal injury after 5-ASA administration. The high frequency of this SNP and the low frequency of the adverse event limits its clinical utility and we cannot recommend its use in guiding treatment choice or monitoring intervals.

Described herein is an analysis of the clinical features of patients with IBD who developed chronic renal damage after administration of 5-ASA compounds. We have conducted the first ever association study of drug-induced renal injury and identify a genome wide association with a class II allele. The rare condition appears to be more common in male patients, can occur after many years of drug administration and even once recognised is only reversible in approximately one third of patients. Although the cost effectiveness of regular renal function measurements has yet to be demonstrated, the potential for serious long-term sequelae should necessitate its regular monitoring.

### REFERENCES FOR EXAMPLE 2

Feagan B G, Macdonald J K. Oral 5-aminosalicylic acid for maintenance of remission in ulcerative colitis. Cochrane database Syst. Rev. 2012; 10:CD000544.
Sandbor W J N, Er S B H. Systematic review: the pharmacokinetic profiles of oral mesalazine formulations and mesalazine pro-drugs used in the management of ulcerative colitis. 2003: 29-42.
Chester A C, Diamond L H, Schreiner G E. Hypersensitivity to salicylazosulfapyridine: renal and hepatic toxic reactions. Arch. Intern. Med. 1978; 138:1138-9.
Calviño J, Romero R, Pintos E, et al. Mesalazine-associated tubulo-interstitial nephritis in inflammatory bowel disease. Clin. Nephrol. 1998; 49:265-7.
Dwarakanath a D, Michael J, Allan R N. Sulphasalazine induced renal failure. Gut 1992; 33:1006-7.
Alivanis P, Aperis G, Lambrianou F, et al. Reversal of refractory sulfasalazine-related renal failure after treatment with corticosteroids. Clin. Ther. 2010; 32:1906-10.
Popoola J, Muller a F, Pollock L, et al. Late onset interstitial nephritis associated with mesalazine treatment. BMJ 1998; 317:795-7.
Arend L J, Springate J E. Interstitial nephritis from mesalazine: case report and literature review. Pediatr. Nephrol. 2004; 19:550-3.
UK Committee on Safety of Medicines. Current Problems No 30: Nephrotoxicity associated with mesalazine (Asacol); 1990: 2.
Muller a F, Stevens P E, McIntyre a S, et al. Experience of 5-aminosalicylate nephrotoxicity in the United Kingdom. Aliment. Pharmacol. Ther. 2005; 21:1217-24.
Gisbert J P, Luna M, Gonzalez-Lama Y, et al. Effect of 5-aminosalicylates on renal function in patients with inflammatory bowel disease: 4-year follow-up study. Gastroenterol. Hepatol. 2008; 31:477-484.
Staa T P Van, Travis S, Leufkens H G M, et al. 5-aminosalicylic acids and the risk of renal disease: a large British epidemiologic study. Gastroenterology 2004; 126:1733-9.
Contreras J L, Floratos A, Holden A L. The International Serious Adverse Events Consortium's data sharing model. 2013; 31:17-19.
Daly A K, Donaldson P T, Bhatnagar P, et al. HLA-B*5701 genotype is a major determinant of drug-induced liver injury due to flucloxacillin. Nat. Genet. 2009; 41:816-819.
Heap G A, Weedon M N, Bewshea C M, et al. HLA-DQA1-HLA-DRB1 variants confer susceptibility to pancreatitis induced by thiopurine immunosuppressants. Nat. Genet. 2014; epub.
Gallagher R M, Kirkham J J, Mason J R, et al. Development and inter-rater reliability of the Liverpool adverse drug reaction causality assessment tool. PLoS One 2011; 6:e28096.
Oliphant A, Barker D L, Stuelpnagel J R, et al. BeadArray technology: enabling an accurate, cost-effective approach to high-throughput genotyping. Biotechniques 2002; Supp1:56-8, 60-1.
Goldstein J I, Crenshaw A, Carey J, et al. zCall: a rare variant caller for array-based genotyping: genetics and population analysis. Bioinformatics 2012; 28:2543-5.
Barrett J C, Hansoul S, Nicolae D L, et al. Genome-wide association defines more than 30 distinct susceptibility loci for Crohn's disease. Nat Genet 2008; 40:955-962.
Anderson C A, Boucher G, Lees C W, et al. Meta-analysis identifies 29 additional ulcerative colitis risk loci, increasing the number of confirmed associations to 47. Nat. Genet. 2011; 43:246-252.
Yang J, Lee S H, Goddard M E, et al. GCTA: a tool for genome-wide complex trait analysis. Am. J. Hum. Genet. 2011; 88:76-82.
Manichaikul A, Mychaleckyj J C, Rich S S, et al. Robust relationship inference in genome-wide association studies. Bioinformatics 2010; 26:2867-73.
Howie B, Fuchsberger C, Stephens M, et al. Fast and accurate genotype imputation in genome-wide association studies through pre-phasing. Nat. Genet. 2012; 44:955-959.
Jia X, Han B, Onengut-Gumuscu S, et al. Imputing amino acid polymorphisms in human leukocyte antigens. PLoS One 2013; 8:e64683.
Li Y, Willer C J, Ding J, et al. MaCH: using sequence and genotype data to estimate haplotypes and unobserved genotypes. Genet. Epidemiol. 2010; 34:816-34.
Purcell S, Neale B, Todd-Brown K, et al. PLINK: a tool set for whole-genome association and population-based linkage analyses. Am. J. Hum. Genet. 2007; 81:559-575.
World M J, Stevens P E, Ashton M A, et al. Nephrology Dialysis Transplantation Mesalazine-associated interstitial nephritis. 1996: 614-621.
Manenti L, Rosa A De, Buzio C. Mesalazine-associated interstitial nephritis: twice in the same patient. Nephrol Dial Transpl. 1997; 12:2031.
Mahmud N, O'Toole D, O'Hare N, et al. Evaluation of renal function following treatment with 5-aminosalicylic acid derivatives in patients with ulcerative colitis. Aliment. Pharmacol. Ther. 2002; 16:207-15.
Patel H, Barr A, Jeejeebhoy K N. Renal effects of long-term treatment with 5-aminosalicylic acid. Can. J. Gastroenterol. 2009; 23:170-6.
Waters A M, Zachos M, Herzenberg A M, et al. Tubulointerstitial nephritis as an extraintestinal manifestation of Crohn's disease. Nat. Clin. Pract. Nephrol. 2008; 4:693-7.
Bijol V, Mendez G P, Nose V, et al. Granulomatous interstitial nephritis: a clinicopathologic study of 46 cases from a single institution. Int J Surg Pathol 2006; 14:57-63.
Izzedine H, Simon J, Piette A, et al. Primary chronic interstitial nephritis in Crohn's disease. Gastroenterology 2002; 123:1436-1440.
Colvin R B, Traum A Z, Taheri D, et al. Granulomatous interstitial nephritis as a manifestation of Crohn disease. Arch. Pathol. Lab. Med. 2014; 138:125-7.
Kornbluth A, Sachar D B. Ulcerative colitis practice guidelines in adults: American College Of Gastroenterology, Practice Parameters Committee. Am. J. Gastroenterol. 2010; 105:501-23; quiz 524.
Dignass A, Assche G Van, Lindsay J O, et al. The second European evidence-based Consensus on the diagnosis and management of Crohn's disease: Current management. J. Crohns Colitis 2010; 4:28-62.
Mowat C, Cole A, Windsor A, et al. Guidelines for the management of inflammatory bowel disease in adults. Gut 2011; 60:571-607.
Siddique N, Farmer C, Muller A F. Do gastroenterologists monitor their patients taking 5-amino-salicylates following initiation of treatment. Frontline Gastroenterol. 2014: flgastro-2014-100452-.
Hestbech J, Hansen H E, Amdisen A, et al. Chronic renal lesions following long-term treatment with lithium. Kidney Int. 1977; 12:205-13.
Hansen H E, Hestbech J, Sorensen J L, et al. Chronic interstitial nephropathy in patients on long-term lithium treatment. Q. J. Med. 1979; 48:577-591.
MARKOWITZ G S, RADHAKRISHNAN J, KAMBHAM N, et al. Lithium Nephrotoxicity: A Progressive Combined Glomerular and Tubulointerstitial Nephropathy. J. Am. Soc. Nephrol. 2000; 11:1439-1448.
Mackensen F, David F, Schwenger V, et al. HLA-DRB1*0102 is associated with TINU syndrome and bilateral, sudden-onset anterior uveitis but not with interstitial nephritis alone. Br. J. Ophthalmol. 2011; 95:971-5.
Satsangi J, Silverberg M S, Vermeire S, et al. The Montreal classification of inflammatory bowel disease: controversies, consensus, and implications. Gut 2006; 55:749-53.

TABLE 2
Montreal classification of disease location
and severity in the two years prior to development of
nephrotoxicity in 151 5-ASA nephrotoxicity cases

| Ulcerative Colitis (n = 93) | | | | Crohn's Disease (n = 58) | | | |
|---|---|---|---|---|---|---|---|
| | Extent | | Severity | | Location | | Behaviour |
| E1 | 5.5% | S0 | 14.0% | L1 | 5.2% | B1 | 81.1% |
| E2 | 20.9% | S1 | 36.6% | L2 | 56.9% | B2 | 17.0% |
| E3 | 69.2% | S2 | 45.2% | L3 | 37.9% | B3 | 1.9% |
| Ex | 4.4% | S3 | 4.3% | L4 | 0.0% | | |

Ulcerative Colitis classification⁴³:
E1-Ulcerative Proctitis;
E2-Left sided UC;
E3-Extensive UC;
Ex-Unknown
S0-Clinical remission;
S1-Mild UC;
S2-Moderate UC requiring steroid or immunomodulator;
S3-Severe UC requiring admission or colectomy
Crohn's disease classification⁴³:
L1-Ileal;
L2-Colonic;
L3-Ileocolonic;
L4-Isolated upper B1-Non-stricturing, non-penetrating;
B2-Stricturing;
B3-Penetrating

TABLE 3
Top GWAS association signals from the combined GWAS and HLA Imputation analysis.

| | | | | | Control | | | | |
|---|---|---|---|---|---|---|---|---|---|
| | | | | | risk | Risk | | OR | |
| | | | Position | Effect | allele | allele | OR | (95% | |
| SNP | Cohort | Chr | (hg19) | Allele | Freq. | Freq. | (SE) | CI) | P value |
| rs3135356 | All | 6 | 32391516 | A | 0.17 | 0.29 | 2.00 | | 1 × 10⁻⁷ |
| | | | | | | | (0.13) | | |
| | Biopsy | | | | | 0.39 | | 3.11 | 4 × 10⁻⁹ |
| | only | | | | | | | (0.19) | |
| rs12204929 | All | 6 | 119396266 | T | 0.05 | 0.11 | 2.79 | | 4 × 10⁻⁷ |
| | | | | | | | (0.20) | | |
| | Biopsy | | | | | 0.10 | | 2.26 | 0.02 |
| | only | | | | | | | (0.34) | |
| rs10488193 | All | 7 | 12274220 | G | 0.11 | 0.21 | 2.15 | | 3 × 10⁻⁶ |
| | | | | | | | (0.15) | | |
| | Biopsy | | | | | 0.25 | | 2.74 | 1 × 10⁻⁵ |
| | only | | | | | | | (0.23) | |

1. A method of identifying a subject afflicted with, or at risk of developing, 5-aminosalicylate nephrotoxicity (5-ASA nephrotoxicity) comprising:
(a) obtaining a nucleic-acid containing sample from the subject; and
(b) analyzing the sample to detect the presence of at least one genetic marker, or an equivalent to at least one genetic marker, selected from those in Table 1,
wherein the presence of at least genetic marker, or an equivalent to at least one genetic marker, from Table 1 in the sample indicates that the subject is afflicted with, or at risk of, developing 5-ASA nephrotoxicity.

2. The method of claim 1, wherein the genetic marker is any of alleles, microsatellites, SNPs, or haplotypes.
